(19) United States
(12) Patent Application Publication
(10) Pub. No.: US 2012/0087867 A1
(43) Pub. Date: Apr. 12, 2012

(54) Method for the Generation of Nuclear Hyper-Antipolarization in Solids Without the Use of High Magnetic Fields or Magnetic Resonant Excitation

(76) Inventors: Dane R. McCamey, New South Wales (AU); Christoph Boehme, Sandy, UT (US); Johan van Tol, Tallahassee, FL (US); Gavin W. Morley, London (GB)

(21) Appl. No.: 13/000,298

(22) PCT Filed: Jun. 19, 2009
(86) PCT No.: PCT/US09/48037

§ 371 Date: Oct. 27, 2011

Related U.S. Application Data
(60) Provisional application No. 61/074,607, filed on Jun. 20, 2008.

(51) Int. Cl. A61K 49/12; G21K 5/00

(52) U.S. Cl. 424/9.3; 250/492.1

(57) ABSTRACT

A method of inducing nuclear spin hyper-antipolarization in a solid material is disclosed and described. The solid material can be subjected to an ultralow temperature and a magnetic field. The solid material can include donor nuclei and a carrier material while the material also has both a nuclear spin and an electron spin which are coupled sufficiently to allow an Overhauser effect. The solid material can be subjected at the ultralow temperature to a light source for a time sufficient to induce a substantial nuclear spin antipolarization in the solid material and form a nuclear spin hyper-antipolarized material. The ultralow temperature and light source are controlled so as to be sufficient to drive a non-equilibrium nuclear Overhauser effect of hyperfine coupled electron and nuclear spins. The resulting nuclear spin hyper-antipolarized material can be used for a variety of applications such as medical imaging and quantum computing. These materials can be readily formed relatively quickly and are generally stable at room temperatures.

### GOVERNMENT INTEREST

This invention was made with government support under National High Magnetic Field Lab under Grant No. VSP 7300-100. The United States government has certain rights in this invention.

This invention was also made with government support from UK EPSRC under Grant Nos. GR/S23506 and EP/D049717/1. The United Kingdom government may also have certain rights to this invention.

### FIELD OF THE INVENTION

This invention relates to generation of hyper-antipolarized materials in solids with high antipolarization. More specifically, such materials can be formed at relatively low magnetic fields and fast polarization times. Therefore, the present invention relates generally to the fields of physics, quantum physics, and spintronics.

### BACKGROUND OF THE INVENTION

Generating hyperpolarization in condensed matter materials has applications for biological imaging techniques and the initialization of proposed quantum information technologies. A recent invention describing the ex vivo hyperpolarization of imaging agents claims the idea that imaging agents can be hyperpolarized in a setup where low temperatures and very high magnetic fields are established. Once hyperpolarization is established, the imaging agents are removed from the hyperpolarization setup and used for in vivo imaging at room temperature.

In such approaches the hyperpolarization is established either by means of (i) “brute force” meaning by a cooling process to very low temperatures under application of very high magnetic fields leads to a thermal equilibrium polarization which then becomes a non-equilibrium hyperpolarization as the sample is heated up to higher temperatures or (ii) a magnetic resonance induced pumping scheme, referred to as dynamic nuclear polarization in the physics literature.

These two methods for the generation of hyperpolarization are technically very complex and very costly. For magnetic fields achievable at reasonable cost (magnetic fields of approx. 10 Tesla, temperatures around the liquid ⁴He boiling point of approx. 4 Kelvin), the brute force method produces still rather low hyperpolarization (<0.5%) whereas the magnetic resonance driven polarization achieves much higher hyperpolarization (demonstrated for silicon to be about 3%-4%) but requires an extremely expensive setup and much time to achieve this hyperpolarization (of the order of hours).

Phosphorus doped crystalline silicon (Si:P) is a model system for investigating spin effects in the solid state and at the same time is a point defect with great technological importance. Si:P has been used since the beginning of the semiconductor industry in the early 1950's for applications ranging from the ubiquitous (thin film transistors) to the conceptual (single electron transistors). The ability to hyperpolarize the spins in this material is important for a number of its applications. Utilizing the nuclear spin of phosphorus donors as quantum bits relies on the ability to obtain a well characterized initial state, which can be obtained by hyperpolarization. Spin polarized silicon microparticles may also have applications for magnetic resonance imaging techniques, similar to other hyperpolarized systems, such as xenon. Whilst it is reasonably simple to obtain large electron spin polarization, for example by using moderate magnetic fields at liquid ⁴He temperatures, doing the same with nuclear spins is difficult due to their much smaller Zeeman splitting. There are a number of schemes used to obtain nuclear spin polarization in excess of the thermal polarization. Dynamic nuclear polarization using off-resonance radiation has been studied extensively. Complex pulses or adiabatic passage effects may be used to manipulate spin states, leading to large polarizations. Electrical injection of hot carriers has been used to obtain positive polarizations, however this requires electrical contact to the sample. Optical excitation with linearly polarized sub-bandgap light has given small (˜0.25%) polarization of ²⁹Si nuclei in silicon with a natural isotopic abundance. Other materials, such as GaAs, have demonstrated nuclear spin polarization over 25% following pumping with polarized light, although these materials are not biologically compatible.

Therefore, none of the existing techniques provides relatively inexpensive approaches, fast polarization times, or high polarization.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention and they are, therefore, not to be considered limiting of its scope. It will be readily appreciated that the components of the present invention, as generally described and illustrated in the figures herein, could be arranged, sized, and designed in a wide variety of different configurations. Nonetheless, the invention will be described and explained with additional specificity and detail through the use of the accompanying drawings in which:

FIG. 1(a) is a diagrammatical sketch of the energy levels of four spin eigenstates of a phosphorus donor atom in silicon in presence of very high magnetic fields in accordance with one embodiment of the present invention. The dashed arrows indicate allowed transitions with their respective rate coefficients. Γ₁ is for longitudinal relaxation processes, Γ_{CE} for relaxation driven by capture-emission of conduction electrons and Γ_{X} for the Overhauser flip-flop process. The two different nuclear orientations are offset horizontally.

FIG. 1(b) is a diagrammatical sketch of the change from a thermally polarized spin ensemble to a hyperpolarized spin ensemble for Tᵣₑₛ>>Tₛₚᵢₙ, to illustrate qualitatively the polarization process in accordance with one embodiment of the present invention. Note that the spin relaxation processes act continuously (not sequentially as illustrated).

FIG. 2(a) is an ESR spectra with and without illumination in accordance with one embodiment of the present invention. The spectra were measured at T=3 K with fᵣₑₛ=240 GHz, with (top) and without (bottom) illumination by a mercury discharge lamp. The polarization is determined by comparing the areas of the two resonances, obtained by fitting the data with two Gaussian line shapes separated by the phosphorus hyperfine splitting, ΔB=4.17 mT (solid line).

FIG. 2(b) is a graph of nuclear spin polarization as a function of time in accordance with one embodiment of the present invention. The graph shows ³¹P nuclear polarization obtained from EPR spectra, measured as a function of illumination time, at T=3 K. The solid line is a single exponential fit to the data.

FIG. 3(a) is an electrically detected magnetic resonance spectrum in accordance with one embodiment of the present invention.

FIG. 3(b) is a graph of polarization as a function of temperature in accordance with one embodiment of the present invention.

FIG. 3(c) is a graph of polarization as a function of illumination intensity in accordance with one embodiment of the present invention.

### DETAILED DESCRIPTION

The following detailed description of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

### DEFINITIONS

In describing and claiming the present invention, the following terminology will be used.

The singular forms “a,” “an,” and “the” include plural referents unless the context clearly dictates otherwise. Thus, for example, reference to “a donor” includes reference to one or more of such materials and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable can in some cases depend on the specific context.

As used herein, “adjacent” refers to the proximity of two structures or elements. Particularly, elements that are identified as being “adjacent” can be either abutting or connected. Such elements can also be near or close to each other without necessarily contacting each other. The exact degree of proximity can in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

In the present disclosure, the term “preferably” or “preferred” is non-exclusive where it is intended to mean “preferably, but not limited to.” Any steps recited in any method or process claims can be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Invention Description

The present invention provides a method of inducing nuclear spin hyper-antipolarization in a solid material which can be fast and result in high nuclear spin polarization. The solid material can be subjected to an ultralow temperature and a magnetic field. The solid material can include donor nuclei and a carrier material while the material also has both a nuclear spin and an electron spin which are coupled sufficiently to allow an Overhauser effect. The solid material can be subjected at the ultralow temperature to a light source for a time sufficient to induce a substantial nuclear spin antipolarization in the solid material and form a nuclear spin hyper-antipolarized material. The ultralow temperature and light source are controlled so as to be sufficient to drive a non-equilibrium nuclear Overhauser effect of hyperfine coupled electron and nuclear spins.

This new way to achieve hyperpolarization of nuclei is in fact not only a polarization of nuclear spins far above the thermal equilibrium but a negative hyperpolarization (so called hyper-antipolarization) whose applicability to imaging techniques works at least as well as hyperpolarization. The technique is able to produce the hyper-antipolarization without the necessity of a magnetic resonance facility under the same conditions as the brute force method mentioned above polarizations. However, in contrast to the brute force method, the hyper-antipolarization obtained is about two orders of magnitude stronger.

The solid material can be any suitable material which includes a donor material and a host matrix material consistent with the requirements set forth herein. Non-limiting examples of suitable carrier or host material comprise or consists essentially of silicon, germanium, silicon-germanium, gallium-arsenide, and combinations thereof. However, other semiconducting materials can also be suitable. Depending on the particular application, the carrier material can include a pharmaceutically acceptable carrier (e.g. silicon).

Similarly, the donor nuclei can be selected from the group consisting of ⁶Li, ⁷Li, ¹²¹Sb, ¹²³Sb, ³¹P, ⁷⁵As, ²⁰⁹Bi, ¹²³Te, ¹²⁵Te, ⁴⁷Ti, ⁴⁹Ti, ²⁵Mg, ⁷⁷Se, ⁵³Cr, ¹⁹⁷Au, and combinations thereof. In one specific example, the solid material can be a phosphorus doped silicon such that the donor nuclei are ³¹P and the carrier material includes silicon.

The solid material and carrier material can be provided in a form suitable for a particular application. Thus, the carrier material can be a bulk material, thin film, or can be provided as a powder. Powdered material can be particularly suited for delivery to a subject by incorporation into a delivery vehicle such as, but not limited to, gels, injectable solutions, oral delivery solutions, pills, and the like.

As described in more detail below, the ultralow temperature is sufficient to allow non-equilibrium driven Overhauser effect in the solid material. However, as a general guideline, the ultralow temperature can vary from about 0.1 K to about 30 K, such as about 1 K to about 3 K.

Similarly, the magnetic field can have a field strength sufficient to cause nuclear Zeeman splitting energy to exceed the nuclear to donor electron hyperfine interaction energy. In one specific embodiment, the magnetic field can have a field strength sufficient to cause polarization of the donor electron spin of greater than about 50%. In another more specific embodiment of the present invention, the magnetic field has a field strength sufficient to cause polarization of the donor electron spin of greater than about 95%. The actual field strength required can vary, depending on the materials and temperature conditions. However, field strength from about 4 to about 15 Tesla can be suitable, although higher field strength can also be used. In one specific embodiment, the magnetic field can be from about 7 to about 10 Tesla. In one specific aspect, the magnetic marker material can be exposed to magnetic fields of 8-10 Tesla at temperatures of approximately 1-3 Kelvin.

Hot charge carriers are injected into the marker material (e.g. by irradiation with light far above the bandgap, meaning a photon energy of several eV in crystalline silicon or by means of an electrical injection). The injection occurs while the material is subjected to the ultralow temperature and the optional magnetic field. The effective temperature driving the nuclear Overhauser effect of hyperfine coupled electron and nuclear spins is changed to a non-equilibrium value. It is this non-equilibrium Overhauser process which then antipolarizes the nuclear spins—in contrast to nuclear T₁—relaxation processes used for the hyperpolarization in prior similar efforts.

Although a wide variety of light sources can be used, in one embodiment, the light source can have an energy greater than the ultralow temperature. For example, the light source can have an energy from about 1 eV to about 5 eV. In one specific embodiment, the light source is a white light source or a mercury lamp. In one alternative, the charge injection of carriers can be accomplished in bulk materials using electrical injection.

In another specific embodiment of the present invention, the ultralow temperature and light source can be chosen so as to maintain Tᵣₑₛ>Tₛₚᵢₙ, during the time over which the light source is applied. Again, actual times can vary depending on the applied field strength and specific materials; however, the time can often range from about 60 seconds to about 1 hour. For example, for phosphorus doped silicon at 8.5 Tesla and 1.37 K, the time for 68% nuclear anti-polarization is about 500 seconds.

Subsequent to forming the desired degree of hyper-antipolarization, the material can be heated to room temperature for a desired application. During heating, spin polarization can be maintained by mitigating heating rates and optionally applying a moderately low magnetic field. Thus, in one specific embodiment, the step of heating includes maintaining an applied magnetic field of less than 1 Tesla. This can help to stabilize antipolarization during heating. Alternatively, heating can be done under conditions which are substantially free of an applied magnetic field.

As a result of these conditions, the principles of the present invention can result in a nuclear spin hyper-antipolarization which is greater than about 5%, and in many cases greater than about 60%. Although stability can vary, typically, local short-range EM fields will have little impact such that the material will stay polarized for relatively long times at room temperature (e.g. greater than about 1 hour). When kept at lower temperatures stability times increase.

Once the nuclear spin hyper-antipolarized material is formed, it can be further used in a variety of applications. Non-limiting examples of such applications can include medical imaging and initialization of a quantum computer.

Consequently, in accordance with one aspect of the present invention, the hyper-antipolarized material can be administered to a subject. This can be done directly or indirectly through incorporation of the material into a suitable delivery vehicle. In one specific embodiment, the hyper-antipolarized material can be attached to a targeted ligand prior to the step of administering. The targeted ligand can be capable of selectively binding with a desired biological tissue. Such ligands are well known in the medical fields and can be chosen based on the desired target tissues. The ligands can be coupled to the material using any number of coupling methods such as, but not limited to, avidin-biotin coupling, self-assembled (SA) polyethylene glycol (PEG) films, and Poly(acrylic acid) (PAAc) surface treatments applied using graft polymerization.

By incorporating the hyper-antipolarized material into a pharmaceutically acceptable carrier, the material can be introduced into a subject and then imaged, e.g. using MRI techniques. Delivery can be oral, subcutaneous, intravenous, or any other suitable delivery route. Pharmaceutically acceptable carriers will depend on the particular application, ligands, and the mode of delivery. Although far from exhaustive, non-limiting examples of suitable carriers can include water and saline solutions (e.g. lactated or Ringer's solution, dextrose solution). Suitable carriers can also optionally include additives such as, but not limited to, buffers, biocides, active agents, drugs, and the like. Furthermore, a second hyper-antipolarized material which is different from the first can be administered to the subject. The second hyper-antipolarized material can be included in admixture with the first or provided in a separate dosage formulation. Such second dosage formulation can be the same or different from the first, e.g. formulated for oral, intravenous, etc.

In another alternative embodiment, the hyper-antipolarized material can be incorporated into a quantum computer. For example, the donor nucleus(i) or donor electron(s) can comprise quantum bit(s). Such applications may be presented in ultra-low temperatures. Optionally, the carrier material can enclose the quantum bit(s) so as to facilitate incorporation into various components of the computer. The hyper-antipolarized material can be incorporated into a computer in any suitable manner. In one aspect, the material is introduced as a quantum bit, in which the donor nuclear spin is the information carrier—see e.g. Kane, Nature 393, 133 (1998) which is incorporated herein by reference. Polarization is thus a way to initialize the system to a known starting state. The material in which quantum bits are built can also contain nuclear spins, such as in GaAs quantum dots, which are a major source of decoherence, directly impacting the time available for computation. By polarizing the nuclei, it has been shown that these coherence times become longer as described by Reilly, Science (2008) which is also incorporated herein by reference. The method described here can also be used to easily polarize the nuclei, thus increasing the available computation time.

Example and Supporting Theoretical Background

The effect for the example of ³¹P phosphorous nuclear spins in a crystalline silicon host matrix for which hyper-antipolarizations were achieved of more than 68% on very short time scales (a few minutes) in comparison to the hyperpolarization schemes of the prior art. The following discussion can also be similarly applied to other host-donor combinations or systems. Anti-polarization of phosphorus donor nuclei in silicon of up to P=−68% has been demonstrated in accordance with one aspect of the present invention. The scheme used is simple, fast and does not involve resonant manipulation of either the nuclear or electronic spin. Instead, the relative populations are modified using photo-excited carriers, generated using white light, at low temperatures (about ⁴He temperature) and in magnetic fields (˜8.5 T) significantly smaller than those required to obtain an equivalent thermal nuclear spin polarization.

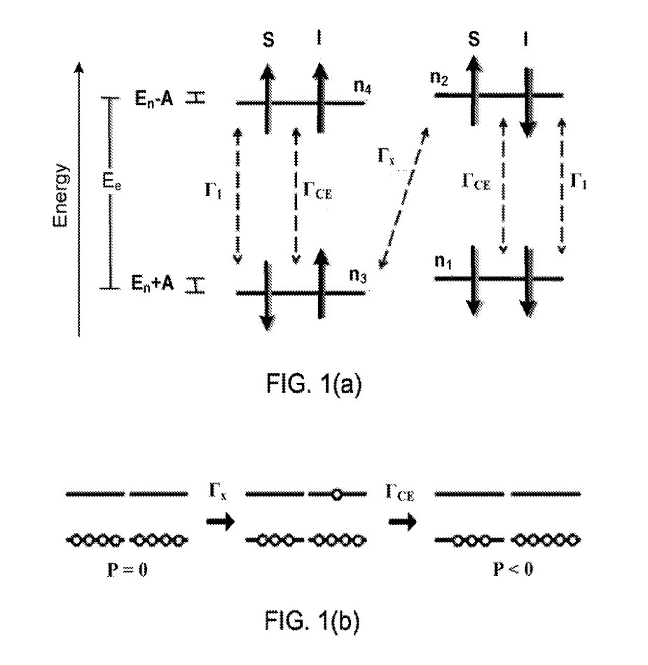

Phosphorus in silicon can be described by the spin (S=1/2) of its donor electron that is coupled to the spin (I=1/2) of the ³¹P nucleus. This model provides a system with four energy levels, as shown in FIG. 1(a) for the presence of strong magnetic fields when the nuclear Zeeman splitting exceeds the nuclear to donor electron hyperfine interaction. At B₀≈8.5 T, the donor electron Zeeman splitting is ΔEₑ≈240 GHz whereas the nuclear Zeeman energy is ΔEₙ≈147 MHz and the hyperfine interaction A=117 MHz. FIG. 1(a) shows the relevant spin relaxation processes that occur in the ³¹P donor atom. The population in each of the four possible spin configurations are labeled n₁ through n₄. Γ₁ is the rate coefficient associated with longitudinal relaxation of the electron magnetization towards thermal equilibrium with the crystal lattice at temperature Tₛₚᵢₙ. Γ_{X} is the rate coefficient associated with the Overhauser spin relaxation process (a flip-flop) between the electron and nuclear spins. The dependence of the Overhauser rate on temperature and magnetic field has been described by Pines et al. who derived an expression

$${T}_{X} = \frac{1}{{Γ}_{X}} = \frac{4 π {ℏ}^{2} {s}^{5} p}{{ω}_{0}^{2} k {T}_{res} {γ}^{2} I {A}^{2}} \qquad ( 1 )$$

where s is the sound velocity of silicon, ρ is the mass density of silicon, γ a multiplicative factor in the range 10 to 100, I the nuclear spin and A the hyperfine constant while ω₀=gμ_{B}B is the Larmor frequency of the electrons with g and μ_{B} representing the electron Landé-factor and Bohr's magneton, respectively and B the applied magnetic field.

It is important to note that the Overhauser relaxation process serves to return the two spin populations n₂ and n₃ to thermal equilibrium with the phonon reservoir, with a temperature Tᵣₑₛ, which is not necessarily the same as the spin temperature Tₛₚᵢₙ. Due to the constant generation of new excess charge carriers by the illumination, a steady state will be established in which a constant density of hot electrons persists. As these hot electrons cascade towards the lattice temperature, they will emit phonons at a constant rate and thus Tᵣₑₛ>Tₛₚᵢₙ. The phonons will also increase Tₛₚᵢₙ, however, this effect is minimal due to the thermal mass of the silicon, which is held constant by the helium bath. Differences between Tᵣₑₛ and Tₛₚᵢₙ have previously been demonstrated using electrical injection of hot carriers. Additionally, the photo-excited carriers may scatter with the bound donor electrons, causing spin relaxation. In contrast to spin relaxation in silicon in the dark, an additional longitudinal relaxation mechanism exists which is driven by the photoexcited electrons. The photoexcited electrons can be captured by a phosphorus donor forming a charged state, with subsequent emission of the extra electron leading to spin relaxation. This process is captured in the rate picture by introducing Γ_{A}(Γ_{B}), the rate coefficient for scattering between spin up (down) free electrons and spin down (up) bound electrons. This capture emission process may be the dominant spin relaxation mechanism of donor electrons, resulting in the donor spins assuming the temperature of the thermalized photocarriers, Tₑ. The electrons which contribute to this process are almost exclusively the thermalized electrons, as the thermalization time is much shorter than the carrier lifetime.

We point out that the temperature that characterizes the spin distribution of the thermalized carriers in semiconductors, Tₑ, is not necessarily the same as Tᵣₑₛ. In Si:P, this leads to a situation where the dominant mechanism for Overhauser relaxation (Γ_{X}) is attempting to move the spin system to a different temperature (Tᵣₑₛ) than the dominant mechanism for electron spin relaxation (Γ_{CE}, Tₑ).

Feher has previously discussed the effect of the phonon reservoir temperature on the polarization of phosphorus in silicon. If the two characteristic temperatures of the present system are equal, Tᵣₑₛ=Tₛₚᵢₙ, then the thermally (hardly) polarized equilibrium population distribution is obtained. However, forcing Tᵣₑₛ>Tₛₚᵢₙ by photoexcitation of charge carriers, the steady state population distribution is changed. The Overhauser process will try to achieve thermal equilibrium between states n₂ and n₃ at a temperature Tᵣₑₛ/and the longitudinal relaxation process will force states (n₁ and n₂) and (n₃ and n₄) to thermal equilibrium at temperature Tₛₚᵢₙ. See FIG. 1(b) for a sketch outlining this process. The result of this situation is that the population of n₁ becomes much larger than the population of all other states, resulting in a net nuclear antipolarization, since

$$P = \frac{( {n}_{1} + {n}_{2} ) - ( {n}_{3} + {n}_{4} )}{( {n}_{1} + {n}_{2} ) + ( {n}_{3} + {n}_{4} )} .$$

Conversely, Tₛₚᵢₙ>Tᵣₑₛ results in nuclear polarization. Spin relaxation of conduction electrons is extremely fast, indicating negligible conduction electron mediated spin interaction between donors. Numerical modeling of this process with realistic values for Tₛₚᵢₙ and Tᵣₑₛ and T₁ indicate that polarization near 100% is achievable.

To demonstrate this effect, electron spin resonance (ESR) and electrically detected magnetic resonance (EDMR) experiments were conducted at B≈8.5 T, corresponding to a resonant frequency, f=240 GHz. The samples used in this demonstration consist of crystalline silicon with (111) surface orientation and a phosphorus doping density [P]˜10¹⁵ cm⁻³, with aluminum surface contacts to allow EDMR.

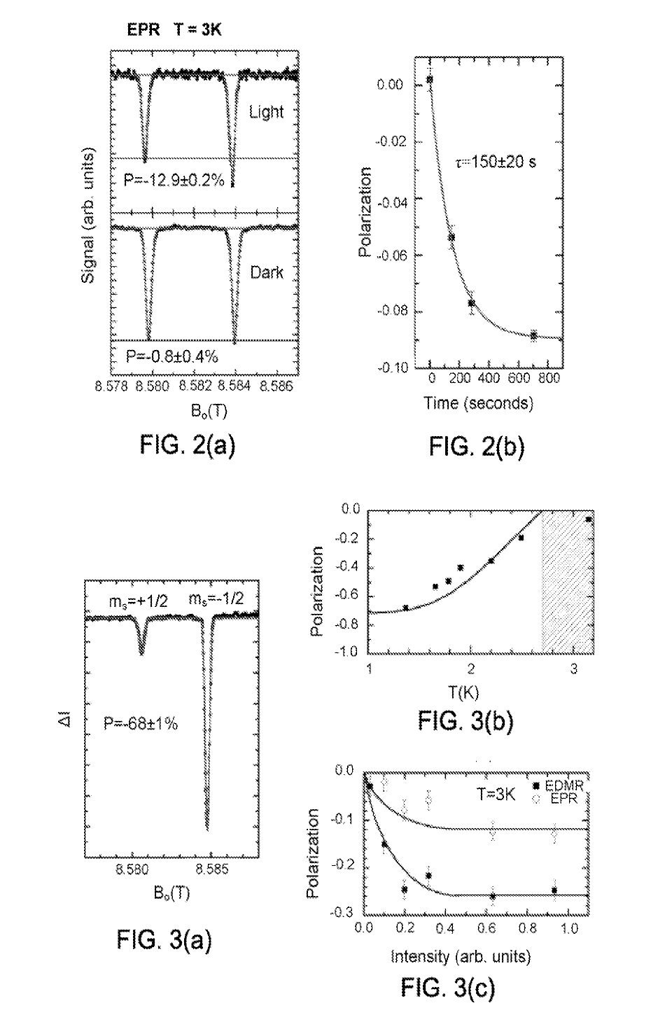

FIG. 2(a) shows two ESR spectra recorded at B≈8.5 T and T=3 K. The spectra were recorded by sweeping B₀ through the expected resonance fields. The two observed resonances were fit with two Gaussian line shapes. Both the g-factor and hyperfine splitting of 4.17 mT confirm the signal is from phosphorus donor electrons. The low-field (high-field) resonance is due to nuclear spins aligned (↑) [anti-aligned(↓)] with the external field. The resonances are saturated due to the long relaxation times, however, it is assumed that the relaxation times are the same and, as a result, can take the area of the resonance as a measure of the number of spins that contribute to it. The polarization of the sample can be determined according to

$$P = \frac{( ↑ - ↓ )}{( ↑ + ↓ )} .$$

The lower spectrum was recorded in the dark, and shows a nuclear polarization P=−0.008±0.004. Next, light from a mercury discharge lamp was shone onto the top side of the sample through an optical fiber, and the ESR spectra was remeasured (upper spectrum). Again, two resonances are visible, however, they have different intensities. Here, the nuclear spin polarization was determined as P=−0.129±0.002. This is a change in polarization over the expected thermal polarization by a factor η=P/P₀≈−78. A similar result is obtained sweeping B₀ in the opposite direction, indicating that the polarization is not a passage effect.

The polarization model discussed above predicts that the time taken to reach a steady-state polarization should be limited by the Overhauser rate, since 1/T_{X}=Γ_{X}<<Γ₁, Γ_{A}, Γ_{B}. By using previously measured low magnetic field (B≈340 mT) values for T_{X}, and extrapolating to the field used in the experiments presented here using Equation 1, the Overhauser time was obtained as T_{X}≈65 s, for Tᵣₑₛ=3K and ω₀=240 GHz. FIG. 2(b) shows the polarization measured via ESR after light was applied to the sample. The data shows a gradual approach to a non-equilibrium steady state. The fit of these data with a single exponential decay function shows excellent agreement and yields a time constant of τ=150±20 s. This is in very good agreement with the predictions of the Overhauser rate made by Pines et al., given the uncertainty of the low field value (˜30 hours) at a higher donor density, and the extrapolation over nearly two orders of magnitude of the magnetic field on which the Overhauser rate depends quadratically.

One aspect of the experiment above suggests that the polarization measured with ESR poses a lower limit on the maximum polarization obtained. ESR measures the polarization in the entire sample; however, only the surface is illuminated. Without being bound to any particular theory, it is expected that, whilst the charge carriers will diffuse throughout the sample, they will thermalize while they diffuse. This will lead to a strong depth inhomogeneity of the reservoir temperature and hence a depth dependence of the polarization. While the polarization will be biggest near the surface which is being illuminated it will be minimized on the opposite sample surface. As background to this thermalization, electrons with a temperature introduced to a material with a different temperature will eventually reach the temperature of the material into which they are introduced, e.g. thermalisation. This happens over a characteristic time called the thermalization time. In the present invention, thermalization happens via the emission of phonons. As the electrons are generated near the surface, they emit phonons as they diffuse through the wafer such that electrons deeper in the wafer will have less energy to give off as phonons, leading to a depth dependence of Tᵣₑₛ.

EDMR is a magnetic resonance detection scheme which is sensitive to spins close to the illuminated sample surface. EDMR relies on the current through a sample being influenced by the observed spin state. In Si:P at high magnetic fields, EDMR is observable due to a spin dependent capture/emission mechanism described by others, which has been included in our polarization model with Γ_{A} and Γ_{B}. The effect of this process is to decrease the current through the sample when resonant excitation of the donor electrons occurs. To measure EDMR, free charge carriers can be used, which are provided by the illumination used to polarize the nuclear spins. FIG. 3(a) shows an EDMR spectrum recorded at T=1.37 K, the lowest temperature achievable with the available equipment in the lab. The spectrum was measured with illumination by a xenon discharge lamp, and a device current, I_{SD}=500 nA. The microwaves were chopped at a frequency of 908 Hz, and the change in current was recorded with a lock-in amplifier. As with the ESR measurements, the spectrum is well fit by two Gaussian line-shapes separated by the hyperfine splitting. Again, the area of the resonances was used as a measure of the population in each nuclear spin state. The polarization measured here is P=−68±1%. This corresponds to an enhancement over the equilibrium polarization of η≈190, and to an effective nuclear spin temperature of ˜−5 mK.

EDMR measurements allow the observation of a ³¹P subensemble with a significantly more homogeneous reservoir temperature than the ESR measurements. Thus, one can use the EDMR to test some of the qualitative properties of the polarization model described, namely, the lattice temperature dependence and the illumination intensity (and hence reservoir temperature) dependence of the observed nuclear polarization. FIG. 3(b) shows the ³¹P polarization as a function of the lattice temperature. It is found to increase monotonically below T≈3 K. Based on the rate model presented in FIG. 1, the polarization was calculated using the measured lattice temperature and a constant reservoir (phonon) temperature whose value was chosen to fit the experimental data. The simulation results are also shown in FIG. 3(b). The best fit of the simulated values to the measured values was achieved for Tᵣₑₛ=2.7K, in agreement with the expectation that hyperpolarization vanishes when Tₛₚᵢₙ≈Tᵣₑₛ. The ratio Γ_{CE}/Γ₁≈4 is also in agreement with expectations. Note that there is significant discrepancy between the fit and the data for temperatures above Tₛₚᵢₙ=2.5 K. While the calculated data predicts no polarization, the measured data shows a clear hyperpolarization of P=−6% at 3K. This discrepancy can be attributed to the assumption of a constant Tᵣₑₛ used in the calculation. Note that Tᵣₑₛ≧Tₛₚᵢₙ for these experiments. Hence, the assumption of a constant Tᵣₑₛ ≡2.7 K becomes unrealistic at Tₛₚᵢₙ>2.7 K. Far above (e.g. about 3 K) the temperature 2.7 K, it is expected that Tₑ=Tₚ, and thus no polarization should occur.

In order to further test the polarization model the excitation spectrum of the excess charge carriers was changed from the xenon lamp used for the acquisition of the data in FIG. 3(a) and (b) to a mercury lamp which has a higher spectral temperature. For the latter polarization was measured with both EDMR and ESR at a constant lattice temperature of Tₛₚᵢₙ=3 K. As shown in FIG. 3(c), the EDMR spectra recorded with the mercury lamp yield a significantly higher polarization of up to P=−24% (instead of 6% at Tₛₚᵢₙ=3 K), independently of the intensity over a range of almost one order of magnitude. As expected, at low intensities, when the excess charge carrier densities drop into a range where the Overhauser process is dominated by Tₛₚᵢₙ, the nuclear polarization vanishes and equilibrium appears. The polarization measured with ESR was consistently≈45% of that measured with EDMR, confirming again the inhomogeneity of the reservoir temperature throughout the sample.

Note that while polarization above P=−68% was demonstrated by the above example, the present invention model predicts the possibility of even higher anti-polarization (e.g. over 95%) at lower temperatures and higher optical excitation rates. This is based on numerical modeling with Tₛₚᵢₙ=1 K, Tᵣₑₛ=3 K, and Γ_{A}(Γ_{B})>>Γ₁>>Γₓ.

The technical simplicity of this polarization method enables the invention to be beneficial for a variety of technical applications. For instance, silicon microparticles are biologically inert which makes them prime candidates as contrast agents for in vivo magnetic resonance imaging. The polarization technique presented above can provide the same level of polarization in microparticles as demonstrated above in bulk material. Given room temperature spin lifetimes>20 minutes for ³¹P nuclei in a-Si:H, a disordered material with a bigger defect density and a larger hyperfine interaction than crystalline silicon, polarization lifetimes of over an hour for this material are expected, easily allowing implementation of such experiments. Also, the rapid polarization of ³¹P nuclear spins demonstrated can offer an initialization mechanism for ³¹P in silicon spin qubits.

In conclusion, the data presented above demonstrates that hyper (anti-) polarization of phosphorous donor nuclear spins in crystalline silicon can be achieved rapidly (on the order of a few minutes) by irradiation with above silicon bandgap light at low temperatures and high magnetic fields. Polarization in excess of 68% was demonstrated, and discussed in terms of a model arising from the increased reservoir temperature driven by phonon emission during thermalization of photoexcited carriers. The qualitative predictions of this model for the polarization dependence on lattice temperature, illumination temperature and intensity have been verified.

In general, the present invention technique can use white light from a lamp at reasonable magnetic fields and temperatures to achieve hyper-antipolarization in only a few minutes. Due to the long room temperature spin coherence times, such material can be used as a contrast agent for magnetic resonance imaging. This is useful as small silicon particles can have surface functionalization, which would allow the material to selectively bind to biological sites to be imaged. The material used, silicon, has the advantage that it can be functionalized, providing contrast at specific biological sites. Hyper-antipolarization can also be used to initialize phosphorus nuclear spin qubits in donor based quantum computer architectures.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

1. A method of inducing nuclear spin hyper-antipolarization in a solid material, comprising:
a) subjecting the solid material to an ultralow temperature and a magnetic field, said solid material including donor nuclei and a carrier material and having both a nuclear spin and an electron spin which are coupled sufficiently to allow an Overhauser effect; and
b) subjecting the solid material at the ultralow temperature to a light source for a time sufficient to induce a substantial nuclear spin antipolarization in the solid material, forming a hyper-antipolarized material,
said ultralow temperature and light source being sufficient to drive a non-equilibrium nuclear Overhauser effect of hyperfine coupled electron and nuclear spins.

2. The method of claim 1, wherein the solid material is a phosphorus doped silicon such that the donor nuclei are ³¹P and the carrier material includes silicon.

3. The method of claim 1, wherein the donor nuclei are selected from the group consisting of ⁶Li, ⁷Li, ¹²¹Sb, ¹²³Sb, ³¹P, ⁷⁵As, ²⁰⁹Bi, ¹²³Te, ⁴⁷Ti, ⁴⁹Ti, ²⁵Mg, ⁷⁷Se, ⁵³Cr, ¹⁹⁷Au, and combinations thereof.

4. The method of claim 1, wherein the carrier material comprises silicon, germanium, silicon-germanium, gallium-arsenide, and combinations thereof.

5. The method of claim 1, wherein the carrier material includes a pharmaceutically acceptable carrier.

6. The method of claim 1, wherein the carrier material is a bulk material.

7. The method of claim 1, wherein the carrier material is a powder.

8. The method of claim 1, wherein the light source has an energy greater than the ultralow temperature.

9. The method of claim 8, wherein the light source has an energy from about 1 eV to about 5 eV.

10. The method of claim 8, wherein the light source is a white light source.

11. The method of claim 1, wherein the ultralow temperature is from 0.1 K to about 30 K.

12. The method of claim 1, wherein the magnetic field has a field strength sufficient to cause nuclear Zeeman splitting energy to exceed an interaction energy of the hyperfine coupled electron and nuclear spins.

13. The method of claim 1, wherein the magnetic field has a field strength sufficient to cause polarization of the donor electron spin of greater than about 50%.

14. The method of claim 1, wherein the magnetic field is from about 4 to about 15 Tesla.

15. The method of claim 1, wherein the nuclear spin hyper-antipolarization is greater than about 5%.

16. The method of claim 15, wherein the nuclear spin hyper-antipolarization is greater than about 60%.

17. The method of claim 1, wherein the ultralow temperature and light source are chosen so as to maintain Tᵣₑₛ>Tₛₚᵢₙ during the time.

18. The method of claim 1, further comprising heating the hyper-antipolarized material to substantially room temperature while maintaining the spin polarization.

19. The method of claim 18, wherein the step of heating is substantially free of an applied magnetic field.

20. The method of claim 18, wherein the step of heating includes maintaining an applied magnetic field of less than 1 Tesla.

21. The method of claim 1, wherein the time is about 500 seconds, the ultralow temperature is about 1.37 K, and the magnetic field has a strength of about 8.5 Tesla.

22. A hyper-antipolarized material produced by the method of claim 1.

23. A hyper-antipolarized material comprising a solid material having a substantial spin antipolarization of greater than 5% at room temperature.

24. The material of claim 23, wherein the spin antipolarization is greater than about 50%.

25. A method of using the material of claim 23, comprising administering the hyper-antipolarized material to a subject.

26. The method of claim 25, further comprising attaching the hyper-antipolarized material to a targeted ligand prior to the step of administering such that the targeted ligand is capable of selectively binding with a desired biological tissue.

27. The method of claim 25, wherein the step of attaching further comprises incorporating the hyper-antipolarized material into a pharmaceutically acceptable carrier.

28. A method of using the material of claim 23, wherein the donor nucleus(i) or donor electron(s) comprise quantum bit(s).

29. The method of claim 28, wherein the carrier material encloses the quantum bit(s).
